(19) United States
(12) Patent Application Publication
(10) Pub. No.: US 2015/0351966 A1
(43) Pub. Date: Dec. 10, 2015

(54) VISOR FOR PROTECTING EYES WITHOUT DISTURBING HAIRSTYLE

(71) Applicant: Stephanie Savin McCauley, Delray Beach, FL (US)

(72) Inventor: Stephanie Savin McCauley, Delray Beach, FL (US)

(21) Appl. No.: 14/731,377

(22) Filed: Jun. 4, 2015

Related U.S. Application Data
(60) Provisional application No. 62/007,819, filed on Jun. 4, 2014.

(51) Int. Cl. A61F 9/04; A45D 8/36

(52) U.S. Cl. CPC A61F 9/045; A45D 8/36

(57) ABSTRACT

A visor has a headband and a bill. The headband extends about the sides of a wearer's head. The bill has a rear support beam affixed to the headband and extends anterior to a wearer a distance sufficient to protect a wearer's face and eyes from the sun. The bill is attached to the top of the headband. The headband is ⅛ of an inch or more thick such that the bill visor is elevated sufficiently above a wearer's hair to prevent damage to a hairstyle.

### CROSS-REFERENCE TO RELATED APPLICATIONS

This application is claims priority to U.S. Provisional Application Ser. No. 62/007,819 filed on Jun. 4, 2014, the contents of which are hereby incorporated by reference in their entirety.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

1. Field of Endeavor

The present invention relates to systems and methods for providing protection from the sun for a wearer's eyes and face while preventing damage to the wearer's hairstyle. More particularly, the invention relates to a headband having a visor and configured to prevent impinging upon a wearer's hairstyle.

2. Background Information

It is common for persons exposed to the sun to use a hat to protect their eyes, and also to protect the skin of their face from sun damage. Hats are also used to accessorize an outfit. Unfortunately, one of the problems encountered with wearing a hat is that the wearer's hair style will tend to be affected in an undesirable manner by the hat. Hats, visors, baseball caps and other devices providing shade for eyes and face are well known to impinge upon and deleteriously effect the wearer's hairstyle or hairdo. This effect is commonly referred to as “hat head.” For example, a headband indentation may be formed into the wearers's hair, the hair may be “flattened” by the hat, or the hair may be rearranged by the hat.

In view of the foregoing, there is a need to provide a means for protecting a person's eyes and face from the sun without damaging the wearer's hairstyle.

### BRIEF SUMMARY OF THE INVENTION

Accordingly, the primary object of the present invention is to provide a visor that protects the wearer from strong light such as sunlight but that does not damage a hair style or hairdo.

In greater detail, the invention provides a visor having a bill that is positioned above the wearer's hairstyle and does not impinge upon it or damage it. The bill extends from a headband or hairband having a thickness sufficient to elevate the visor above a hairstyle.

In one embodiment, the bill is convex and supported by an integral peripheral frame, the rear region of which is affixed to the headband.

In another embodiment the visor may have a bill that may be removably attachable to the headband.

In a further embodiment, the headband may include teeth or a comb to engage the wearer's hair.

In a further embodiment, the distance of the bill from the top of a wearer's head may be adjusted.

These and other objects and advantages of the present invention will become apparent from a reading of the attached specification and appended claims. There has thus been outlined, rather broadly, the more important features of the invention in order that the detailed description thereof that follows may be better understood, and in order that the present contribution to the art may be better appreciated. There are features of the invention that will be described hereinafter and which will form the subject matter of the claims appended hereto.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a side view of a visor in accordance with the principles of the invention;

FIG. 2 is a perspective view of a visor in accordance with the principles of the invention;

FIG. 3 is a perspective view of a visor in accordance with the principles of the invention;

FIG. 4 is a rear view of a visor in accordance with the principles of the invention;

FIG. 5 is a bottom plan view of a visor in accordance with the principles of the invention;

FIG. 6 is a cross-sectional view of an alternative embodiment of a visor in accordance with the principles of the invention;

FIG. 7 is a cross-sectional view of an alternative embodiment of a visor in accordance with the principles of the invention.

### DETAILED DESCRIPTION

Before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

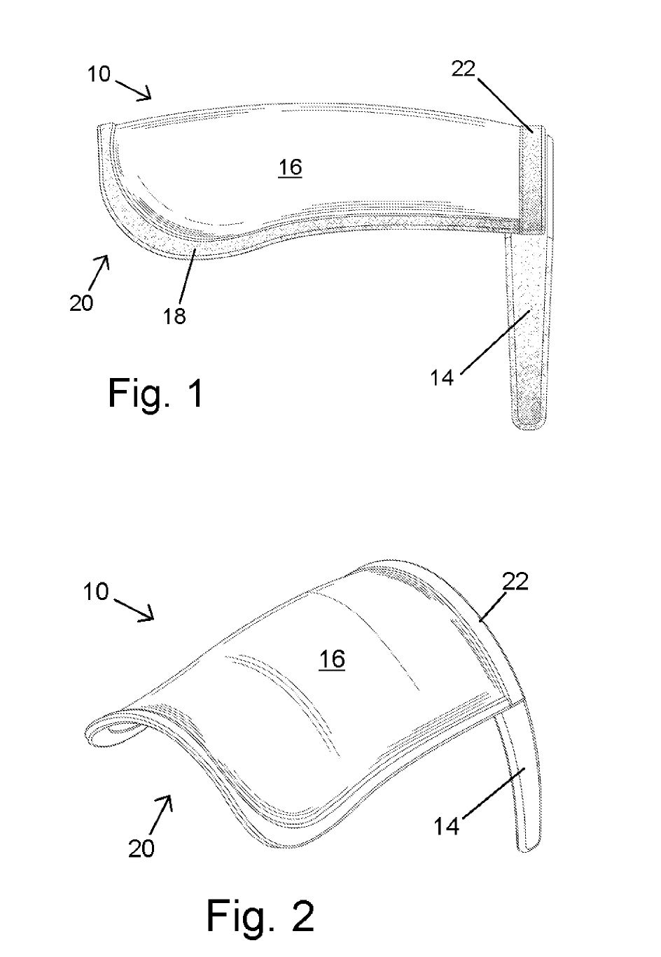

FIG. 1-5 show a visor 10 in accordance with the principles of the invention. The visor 10 may include a headband 14 and a bill 20. The bill 20 may have a panel 16 defined by a peripheral frame 18 and a support beam 22 positioned at the rear of the bill 20 affixed to the top 26, i.e. the exterior side, of the headband 14. The front of the bill 24 may extend in an anterior direction from the support beam 22 a distance sufficient to shade a wearer's face and eyes. The support beam 22 of the bill 20 may be secured to the headband 14. Headband 14 may be relatively thick, extending an eighth of an inch, one quarter of an inch or more from the head 12 of the wearer. As a result, the visor 20 is not in contact with the wearer's hair or only minimally impinges upon a wearer's hairstyle.

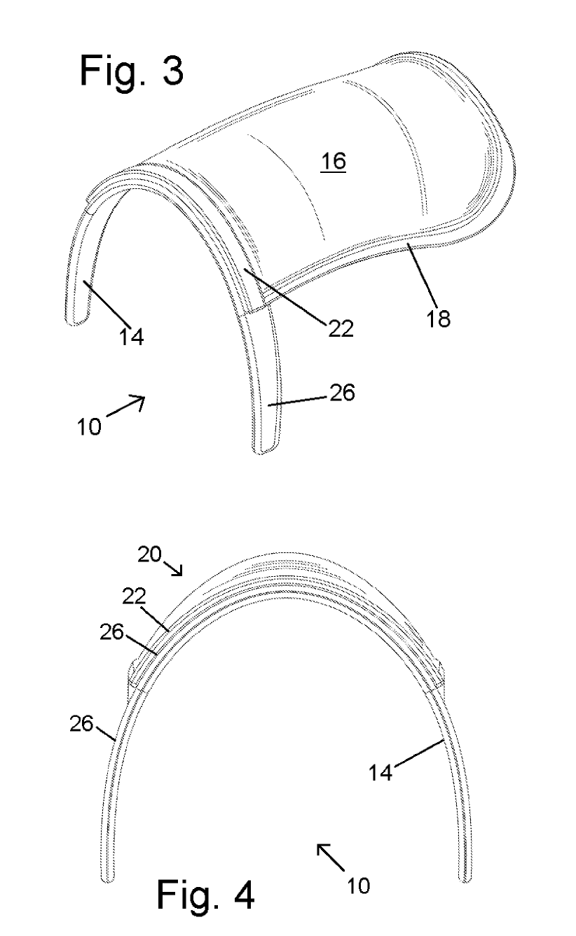

FIGS. 2 and 3 show a visor 10 in accordance with the principles of the invention from two perspective views. In these Figures, it may be seen that the bill 20 has an arcuate, convex shape. The downward extending sides of the visor may provide additional shade and protection to a wearer's face and eyes. The convex shape may also minimize the blocking of a wearer's view. The bill 20 of visor 10 may be shaped more like the bill of a baseball hat than a typical visor. The support beam 22 of the visor 20 may be substantially flush with headband 14 and this may give bill 20 a convex, arcuate shape. The frame 18 may be formed by folding the material of the panel 16 over on itself, similar to a cuff on a shirt or a reinforced seen. Optionally, a semi-rigid device, such as a wire or plastic band may be provided within the frame 18 to provide a desired amount of rigidity and to give shape to the bill 20.

The support beam 22 of visor 20 may be attached to the top 26 of the headband 14 utilizing any desired method, for example, sewing, snapping, tying, gluing or the like. Optionally, the bill 20 may be removably attached to headband 14 such that headband 14 may be used as only a headband or hairband.

FIG. 4 shows a rear view of a visor 10 in accordance with the principles of the invention. Headband 14 has an arcuate shape and is elastic, similar to hairbands and headbands that have been in use for years. Headband 14 may fit about a wearer's head and may be held in place by frictional engagement due to its inherent elasticity. Headband 14 may be thicker than normal headbands such that the top 26 is located ⅛ of an inch, a quarter inch or more above the head of a wearer. Because headband 14 is relatively thick and bill 20 is attached to the top or exterior 26 of the headband, the visor 20 extends over and above a wearer's hair instead of impinging upon it and mashing it down.

Optionally, the bill 20 may be attached to the headband 14 by a structure that extends upward from headband 14. Such a structure may optionally be adjustable to accommodate hairstyles of different thicknesses.

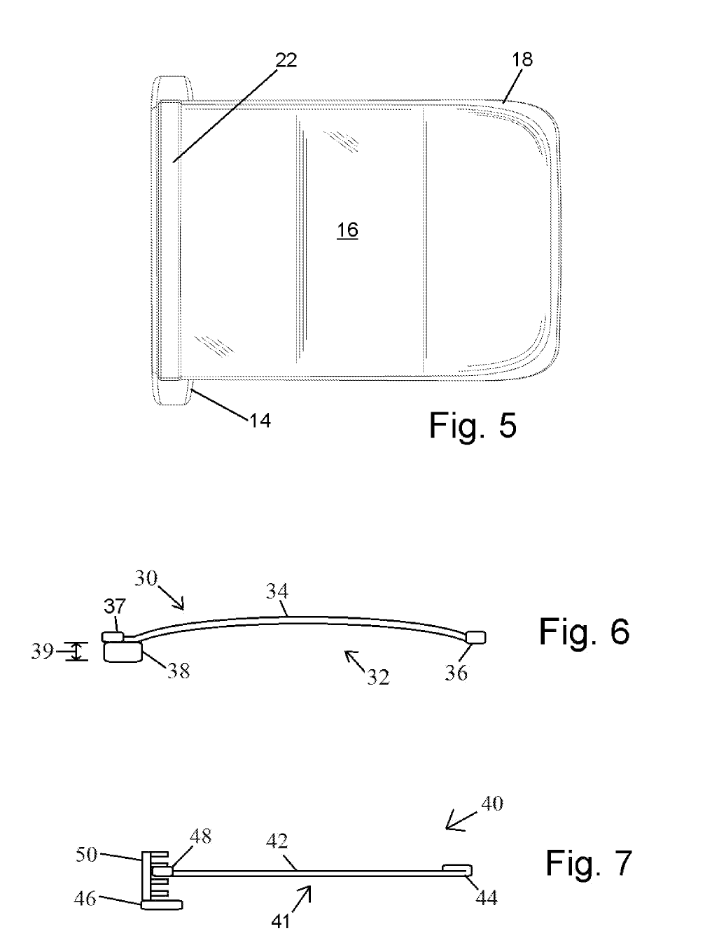

FIG. 5 shows a bottom plan view of a visor 10 in accordance with the principles of the invention. Frame 18 may be substantially U-shaped. The frame 18 may be semi-rigid such that the bill 20 substantially maintains its shape. Optionally, the bill 20 may have more of an oval or rectangular shape. The headband 14 may be a substantially plain headband as shown here, or may optionally include teeth protruding rearward or downward such that it functions as a hairband and may be used to facilitate certain hairstyles in addition to protecting hairstyle. Because only the hairband 14 impinges upon the hair and the bill 20 extends over the hair and does not substantially impinge upon a wearer's hair, a wearer's hairstyle is not damaged or mussed by wearing the visor 10.

FIG. 6 shows a cross-sectional view of an alternative embodiment of a visor 30 in accordance with principles of the invention. In this embodiment, the visor 30 has a bill 32 attached to a headband 38. Visor 32 is comprised of a convex panel 34, a peripheral frame 36 and a support beam 37. Frame 36 and support beam 37 may be semi-rigid or rigid and may cause the bill 32 to remain in a desired shape and position. The support beam 37 may be attached to headband 38. Headband 38 may be sized to have a thickness 39 sufficient to allow the visor 32 two extend substantially above a hairstyle without damaging it. In this embodiment, the panel 34 of visor bill is curved and slightly convex along its longitudinal axis as well as along its transverse axis as shown in FIGS. 1 and 2. Optionally, the degree of curvature along either a longitudinal and transverse axis may be adjustable.

FIG. 7 shows another alternative embodiment of a visor 40 in accordance with principles of the invention. The visor 40 may be comprised of a bill 41 having panel 42 whose perimeter may be folded over in order to make a semi-rigid frame 44. Optionally, the frame 44 may include a wire or plastic band to increase rigidity. The support beam 48 may include an attachment device 48 that may attach to connector 50 extending upward from the headband 46 in one or more locations. Use of connector 50 and attachment mechanism 48 may allow an operator to adjust the distance between a wearer's hairstyle and the bill 41. This may allow the visor 40 to accommodate a variety of different hairstyles without impinging upon them or otherwise damaging or modifying them.

Whereas, the present invention has been described in relation to the drawings attached hereto, it should be understood that other and further modifications, apart from those shown or suggested herein, may be made within the spirit and scope of this invention. Descriptions of the embodiments shown in the drawings should not be construed as limiting or defining the ordinary and plain meanings of the terms of the claims unless such is explicitly indicated.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

1. An improved sun visor comprising:
a headband having a top side, wherein the headband has a thickness of at least ⅛ of an inch; and,
a bill having a panel defined by a peripheral frame and a rear support beam;
wherein the rear support beam is affixed to the top side;
wherein the bill is a distance above the head of a wearer sufficient to prevent the bill from substantially impinging upon the hair of said wearer.
